(19) United States
(12) Patent Application Publication
(10) Pub. No.: US 2005/0096549 A1
(43) Pub. Date: May 5, 2005

(54) Techniques for transperineal delivery of a denervating agent to the prostate gland

(75) Inventors: Martin T. Gerber, Maple Grove, MN (US); John M. Swoyer, Andover, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/698,213

(22) Filed: Oct. 31, 2003

(51) Int. Cl. A61B 8/14

(52) U.S. Cl. 600/464

(57) ABSTRACT

The invention is directed to a technique for delivering a denervating agent to a patient's prostate gland. In particular, the invention is directed to a transperineal technique for delivering the denervating agent. Devices and systems are also described for use in implementing the technique.

### FIELD OF THE INVENTION

The invention relates generally to prostate treatment and, more particularly, to techniques for a delivering an agent to the prostate gland.

### BACKGROUND

Benign prostatic hyperplasia (BPH) is one of the most common medical problems experienced by men over 50 years old. Urinary tract obstruction due to prostatic hyperplasia has been recognized since the earliest days of medicine. Hyperplastic enlargement of the prostate gland often leads to compression of the urethra, resulting in obstruction of the urinary tract and the subsequent development of symptoms including frequent urination, decrease in urinary flow, nocturia, pain, discomfort, and dribbling.

One common surgical procedure used for treating BPH is transurethral needle ablation (TUNA). The TUNA technique involves transurethral delivery of an electrically conductive ablation needle to the prostate site. The electrically conductive ablation needle penetrates the prostate gland in a direction generally perpendicular to the urethral wall, and delivers electrical current to ablate prostate tissue. The electrical current heats tissue surrounding the ablation needle tip to destroy prostate cells, and thereby create a lesion within the prostate gland. The destroyed cells may be absorbed by the body, infiltrated with scar tissue or become non-functional.

Other transurethral ablation procedures involve delivery of microwave, radio frequency, acoustic, and light energy to the prostate gland. These procedures, as well as the TUNA procedure, involve tissue trauma that can be painful for the patient. For these and other reasons, alternative techniques for treating BPH may be desirable for some patients.

U.S. Pat. No. 6,551,300 to McGaffigan discloses a transurethral ablation device that delivers a topically applied anesthetic agent gel to a urethral wall. U.S. Published Patent Application no. 2002/0183740 to Edwards et al. discloses a transurethral ablation device to ablate prostate tissue via electrically conductive needles. U.S. Pat. No. 6,241,702 to Lundquist et al. describes another transurethral ablation needle device. U.S. Pat. No. 6,231,591 describes instruments for localized delivery of fluids to a portion of body tissue, including the prostate. U.S. Pat. No. 6,537,272 to Christopherson et al. describes creation of a virtual electrode by delivery of a conductive fluid to a tissue site.

U.S. Pat. No. 6,365,164 to Schmidt and U.S. Patent publication 2002/0025327 disclose the use of neurotoxin therapy for treatment of urologic and related disorders. Table 1 below lists various documents that disclose either devices for transurethral ablation of prostate tissue or techniques for neurotoxin delivery to treat urologic disorders.

TABLE 1

| U.S. Pat. No. | Inventors | Title |
|---|---|---|
| 2002/0183740 | Edwards et al. | Medical probe device and method |
| 6,551,300 | McGaffigan | Device and method for delivery |
| | | of topically applied local |
| | | anesthetic to wall forming a |
| | | passage in tissue |
| 6,241,702 | Lundquist et al. | Radio frequency ablation device |
| | | for treatment of the prostate |
| 6,231,591 | Desai | Method of localized fluid therapy |
| 6,537,272 | Christopherson et al. | Apparatus and method for creating, |
| | | maintaining, and controlling a |
| | | virtual electrode used for the |
| | | ablation of tissue |
| 6,365,164 | Schmidt | Use of neurotoxin therapy for |
| | | treatment of urologic and |
| | | related disorders |
| 2002/0025327 | Schmidt | Use of neurotoxin therapy for |
| | | treatment of urologic and |
| | | related disorders |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously in order to exploit techniques of the present invention.

### SUMMARY OF THE INVENTION

The invention is directed to techniques for delivering a denervating agent to a patient's prostate gland. In particular, the invention is directed to a transperineal technique for delivering the denervating agent. Devices and systems are also described for use in implementing the technique.

The invention has certain objects. That is, various embodiments of the present invention provide solutions to one or more problems existing in the prior art with respect to treatment of benign prostatic hyperplasia (BPH) or other prostate disorders. The problems include, for example, pain and trauma associated with some existing transurethral ablation techniques. In existing techniques, such as the TUNA procedure, electrode needles are deployed into the urethral wall to penetrate prostate tissue to be ablated. The needles deliver energy to ablate prostate tissue and thereby form lesions. Delivery of ablation energy can be traumatic and painful for some patients. In addition, ablation techniques may be difficult to perform for some patients.

Various embodiments of the present invention have the object of solving at least one of the foregoing problems. For example, it is an object of the present invention to overcome at least some of the disadvantages of the ablation procedures. To that end, it is a further object of the invention to provide alternative to an ablation procedure for BPH therapy which may be easier to perform than ablation procedures. As another object, the invention may provide BPH therapies that are less painful to the patient.

Various embodiments of the invention may possess one or more features capable of fulfilling the objects identified above. In general, the invention provides techniques and devices for delivering a denervating agent, such as botulinum toxin, to a patient's prostate gland. In particular, this invention provides a transperineal technique the denervating agent to the patient's prostate gland. Devices and systems are also described for use in implementing the technique.

In the transperineal approach, a method may include inserting an imaging apparatus into a rectum of a patient, generating one or more images of a prostate gland via the imaging apparatus, inserting a needle through a perineum the patient, positioning a distal end of the needle in proximity to the prostate gland based on the one or more images, actuating a mechanism to cause the distal end of the needle to spring bias into the prostate gland, and delivering a denervating agent to the prostate gland via a lumen of the needle.

A system used in the transperineal approach may include an imaging apparatus sized for insertion into a rectum of a patient to generate one or more images of a prostate gland, a needle for insertion through a perineum of the patient in proximity to the prostate gland based on the one or more images, the needle defining a lumen, and a spring mechanism to bias the needle into the prostate gland upon actuation such that a denervating agent can be delivered to the prostate gland through the lumen.

In comparison to known implementations of prostate ablation, various embodiments of the present invention may provide one or more advantages. In particular, the invention provides alternatives to an ablation procedure for treatment of BPH or other prostate disorders which may be easier to perform by a physician and/or less traumatic to the patient.

Moreover, in comparison to known techniques for delivery of neurotoxins, the invention can provide significant improvements. For example, the invention can allow for more precise delivery of a denervating agent to the prostate gland, possibly reducing the amount of the denervating agent needed for effective therapy. The invention can also simplify or improve the delivery of a denervating agent to the prostate gland by reducing the likelihood of complication. For some patients, the transperineal technique described herein may be more effective than alternative techniques, such as transurethral or transrectal techniques also described herein. Relative to transurethral techniques, the transperineal technique also has the advantage of not violating the urethra of the patient.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a device for transurethral delivery of a denervating agent to the prostate gland of a patient.

FIG. 2 is a cross-sectional side view of a distal tip of a shaft of a transurethral denervating agent delivery device according to an embodiment of the invention.

FIG. 3 is perspective top view of a distal tip of a shaft of a transurethral denervating agent delivery device according to another embodiment of the invention.

FIG. 4 is a block diagram of a denervating agent delivery assembly which may be used with one or more devices or systems described herein.

FIG. 5 is a conceptual side view of another denervating agent delivery assembly which may be used with one or more devices or systems described herein.

FIG. 6 is a flow diagram illustrating a transurethral technique for delivering a denervating agent to the prostate gland according to an embodiment of the invention.

FIG. 7 is a flow diagram illustrating a transurethral technique for delivering a denervating agent to the prostate gland according to another embodiment of the invention.

FIG. 8 is a flow diagram illustrating a transurethral technique for delivering a denervating agent to the prostate gland according to another embodiment of the invention.

FIG. 9 is a conceptual cross-sectional side view of a system that may be used in a transperineal technique for delivery of a denervating agent to the prostate gland according to an embodiment of the invention.

FIG. 10 is a flow diagram illustrating a transperineal technique for delivering a denervating agent to the prostate gland.

FIG. 11 is a conceptual cross-sectional side view of a system that may be used in a transrectal technique for delivery of a denervating agent to the prostate gland according to an embodiment of the invention.

FIG. 12 is a flow diagram illustrating a transrectal technique for delivering a denervating agent to the prostate gland.

### DETAILED DESCRIPTION OF THE PREFERRED EMBODIMENTS

Delivery of a denervating agent, such as botulinum toxin, to a patient's prostate gland has shown significant promise as a therapy for treating benign prostatic hyperplasia (BPH) or other prostate disorders. For this reason, techniques and devices that enable the delivery of a denervating agent to a patient's prostate gland are highly desirable. This disclosure describes three different techniques for delivery of a denervating agent to the prostate gland: a transurethral technique, a transperineal technique and a transrectal technique. Various devices and systems are also described for use with the respective techniques, or the like.

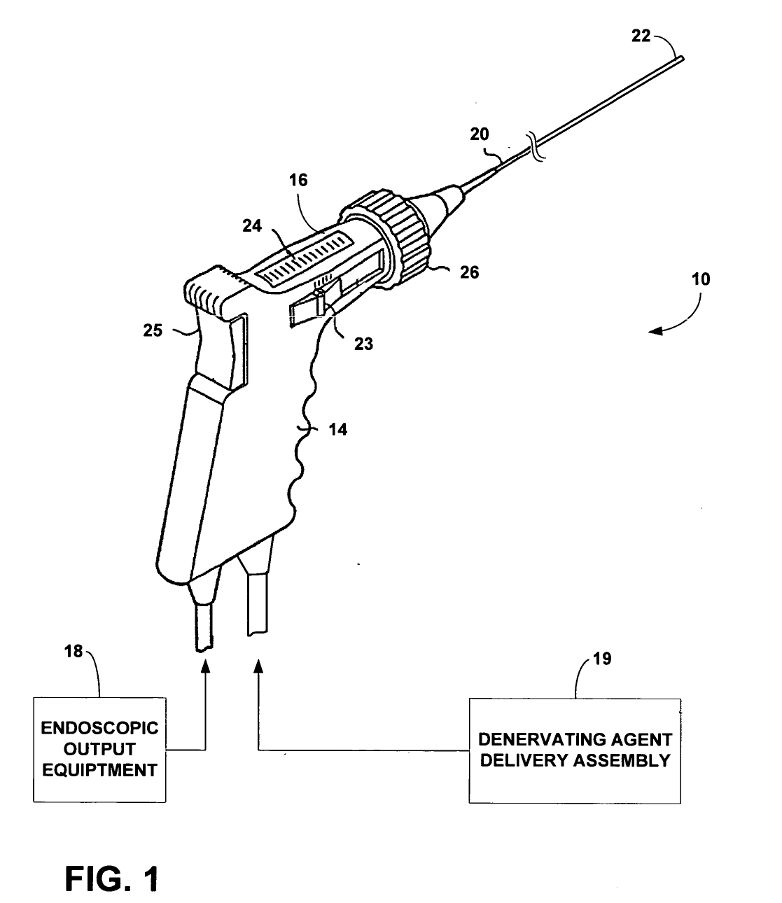

FIG. 1 is a schematic diagram illustrating a device 10 for transurethral delivery of a denervating agent to the prostate gland of a patient. As shown in FIG. 1, device 10 includes a handle 14, a barrel 16, and a transurethral shaft 20 extending from barrel 16. Device 10 also includes a denervating agent delivery assembly 19, which can be viewed as part of device 10 or a separate component that attaches to device 10. In addition, endoscopic output equipment 18 couples to device 10 and can be viewed as part of device 10 or separate equipment. An endoscope may extend through handle 14, barrel 16 and shaft 20 to the distal end 22 of shaft 20 to allow for imaging guidance of shaft 20 to the desired location.

Shaft 20 is sized for insertion into a urethra of a male patient. Shaft 20 may comprise a semi-flexible material such as a plastic or semi-flexible metal housing. As will be described, one or more needles extend from a side of distal end 22 of shaft 20. A denervating agent flows through a lumen of the needle from denervating agent delivery assembly 19 to the targeted sight of the prostate gland, e.g., upon actuation of switch 25. Denervating delivery agent assembly 19 may be configured to provide carefully metered dosages of the denervating agent, and to permit repeated application of such dosages. The dosages may be the same amount for each repeated application. Alternatively, denervating agent delivery assembly 19 may permit selective application of different metered dosages at different times over the course of treatment.

The physician inserts shaft 20 into the urethra of the patient and, using endoscopic output displayed on endoscopic output equipment 18, maneuvers distal end 22 of the shaft into close proximity to the prostate gland of the patient. In addition or as an alternative to endoscopic imaging, fluoroscopic or ultrasonic imaging may be used in some applications. Once distal end 22 is positioned proximate the prostate gland, the physician activates an actuator to cause the needle (not shown in FIG. 1) to extend out from a side of distal end 22 of shaft 20 to pierce the patient's prostate gland. For example, slide bar 23 may operate as an actuator for a spring loaded needle such that when the physician advances slide bar 23, it releases a spring to spring bias the needle into the patient's prostate gland. Such a spring-loaded needle may improve the ability to pierce the prostate gland for delivery of a denervating agent. An indicator 24 may be provided to track advancement of the needle for overhead visibility by the physician. Slide bar 23 may also allow for advancement of the needle to different depths, depending on the particular dose being delivered.

Once the needle is advanced into the prostate gland of the patient, a denervating agent can be delivered to the prostate through a lumen of the needle. For example, a switch 25 or other actuator mechanism can cause the denervating agent to flow from denervating agent delivery assembly 19 through a lumen of the needle to the prostate gland of the patient. In particular, switch 25 may be electrically coupled to activate a pump that actively pumps denervating agent from a reservoir within denervating agent delivery assembly 19. Alternatively, switch 25 may be mechanically coupled to open a valve that permits flow of the denervating agent into the lumen of the needle. In either case, the denervating agent can be easily delivered to the prostate gland for therapeutic purposes. Various embodiments for realizing denervating agent delivery assembly 19 are described in greater detail below.

In some embodiments, multiple needles extend through shaft 22 for simultaneous delivery of a denervating agent to different prostate locations, e.g., to different lobes of the prostate gland. In that case, each of the needles may be coupled to the same denervating agent reservoir or may be coupled to different agent reservoirs to provide more accurate pressurized control over the delivery of the denervating agent via the different needles. Different denervating agents could also be delivered to the different lobes if separate reservoirs are coupled to each of a plurality of needles. Each of the needles may be advanced to depths which are desirable for delivery of the denervating agent at the location corresponding to the given needle.

In other embodiments, a single needle may extend through shaft 22. In that case, however, the same needle may be used to pierce the prostate gland in different locations so that doses of the denervating agent can be delivered to the different locations. Device 10 may include a wheel 26 which permits rotation of shaft 20, e.g., to position the needle at different positions within the urethra with respect to the prostate gland. In that case, the physician may advance slider bar 23 to pierce the prostate gland at a first location and then actuate switch 25 to deliver a first dose of the denervating agent to the prostate gland. The physician can then draw slider bar 23 back to remove the needle from the patient's prostate gland, rotate wheel 26 or otherwise move shaft 20 with respect to the prostate gland, and advance slider bar 23 to pierce the prostate gland at a second location. Accordingly, additional doses of the denervating agent can be delivered to the prostate gland at different locations. If desired, the needle may be advanced to different depths at the different locations.

Advantageously, a plurality of doses can be delivered to the prostate gland at different locations without removing shaft 22 from the urethra. Again, this can be achieved either via multiple agent delivery needles that extend from distal end 22 for simultaneous delivery of the denervating agent to the different locations, or by using a single needle which is advanced and withdrawn from the different prostate locations. For example, the same needle can be used to deliver the denervating agent to a first location, a second location, a third location, a fourth location, and so forth, without withdrawing shaft 22 from the patient's urethra. In any case, by facilitating precise delivery of discrete doses to the prostate gland at different locations, a reduced amount of the denervating agent may become effective in achieving therapeutic results. For example, discrete doses of approximately botulinum toxin may be delivered to the different lobes of the prostate gland for effective therapeutic results. In particular, the discrete doses may comprise 0.3-0.7 milliliter of botulinum toxin, more preferably 0.4-0.6 milliliter of botulinum toxin, and still more preferably 0.5 milliliter of botulinum toxin may comprise a given dose. These dosages may include a diluent of approximately 0.9 percent sodium chloride in saline, resulting in dosages that include between approximately 1.25 to 10 units of botulinum toxin per 0.1 milliliter.

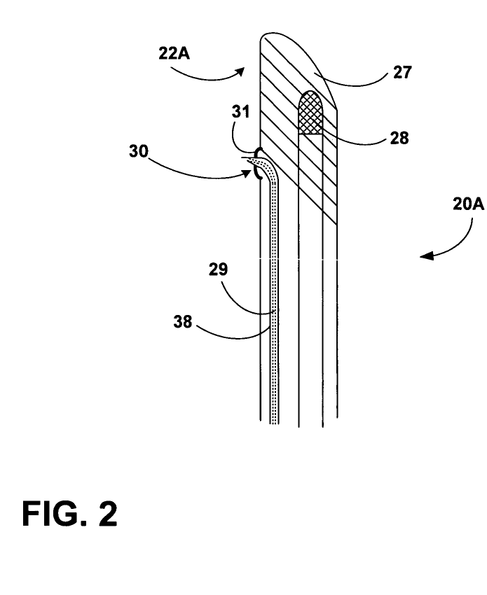

FIG. 2 is a cross-sectional side view of a distal tip 22A of shaft 20A, which may correspond to distal tip 22 of shaft 20 (FIG. 1), in accordance with an embodiment of the invention. In the example of FIG. 2, distal tip 22A is formed with a shape that defines an offset-curvature. Such a shape can aid the physician's guidance of shaft 20A to the desired location adjacent the prostate gland. In particular, an offset-curvature similar to that shown on distal tip 22A can improve the ability of a physician to maneuver shaft 20A through the urethra into proximity to the prostate gland. The distal tip my define a diameter of approximately 3 to 7 millimeters, with a distal-most point being offset from the center axis of shaft by approximately 5 to 20 percent of the diameter. Again, such an offset curvature at distal tip 22A can improve the ability to maneuver and navigate shaft into proximity to the prostate gland.

In addition, shaft 20A may include a substantially transparent or translucent section 27 on or near distal tip 22A. An endoscopic 28 may be housed in or near translucent section 27 such that endoscope 28 is hermetically sealed from the environment, but can visualize the environment through section 27. Accordingly, images can be taken by endoscopic 28 as the physician navigates distal tip 22A of shaft 20A in proximity to the prostate gland. In one example, endoscope 28 comprises a cystoscope such as those commonly used for urinary tract viewing.

Needle 38 defines a lumen 29 through which a denervating agent can be delivered to the prostate site. Needle 38 deflects from a side of shaft 20A through hole 30 when the physician advances slider bar 23 (FIG. 1). Again, needle 38 may be spring-loaded in that slider bar 23 spring biases needle 38 out of hole 30 very quickly, in order to bias needle 38 against the prostate tissue and improve the ability to pierce the prostate gland. Also, needle 38 may be advanced to different depths. A fluid connection hub may facilitate attachment of needle 38 to denervating agent delivery assembly 19 (FIG. 1).

Hole 30 may be sealed by an optional silicone seal 31 or another suitable sealing mechanism to avoid ingress of fluid into shaft 20A prior to extension of needle 38 outward from shaft 20A. In addition, seal 31 may be advantageous to limit residual amounts of the denervating agent in lumen 29 from exiting hole 30, e.g., as shaft 20A is removed from the patient's urethra.

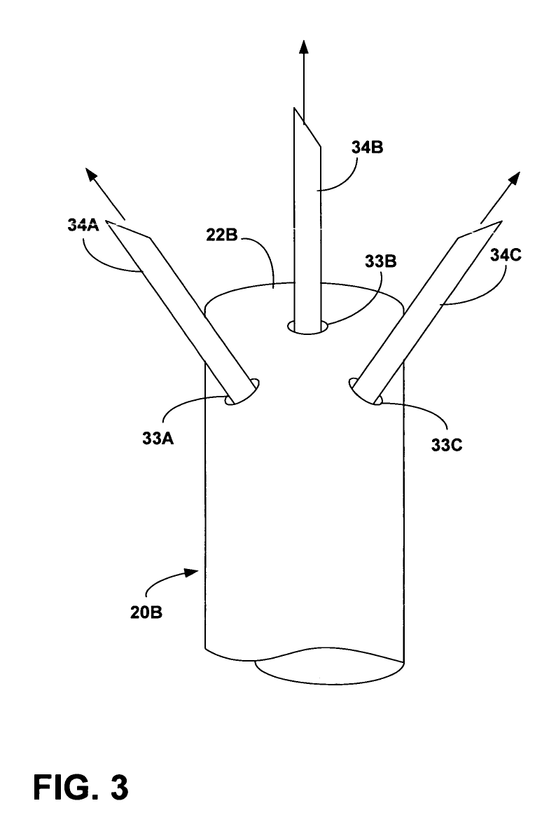

FIG. 3 is a perspective top view of a distal tip 22B of shaft 20B, which may correspond to distal tip 22 of shaft 20 (FIG. 1), in accordance with another embodiment of the invention. In the example of FIG. 3, distal tip 22A defines a plurality of holes 33A, 33B and 33C. A plurality of needles 34A, 34B, 34C are extendable through holes 33. Each of needles 34 defines a lumen for delivery of the denervating agent. For example, when the physician advances slider bar 23 (FIG. 1), each of needles 34 may extend from a side of shaft 20B at a location proximate to distal tip 22B. The movement of needles 34 may be defined to correspond to specific angular positions associated with prostate glad locations, e.g., specific lateral and medial lobes of the prostate gland where delivery of the denervating agent is desired. In other words, needles 34 may protrude from holes 33 to locations that correspond to locations of prostate lobes of a typical human-male anatomy.

Although not illustrated in FIG. 3, distal tip 22B may define one or more other features described above with reference to FIG. 2, such as an offset curvature to aid guidance of shaft 22B, a translucent section, and endoscope housed in the translucent section, seals over holes 33, and so forth. In addition, each of needles 34 may be spring-loaded, as described herein, in order to improve the ability of needles 34 to pierce the prostate gland. Also, each of needles 34 may be advanced to different depths, either collectively or individually.

Each of needles 34 may deliver the denervating agent independently in response to actuation of a unique actuator. Alternatively, needles 34 may deliver the denervating agent simultaneously upon actuation of a common actuator (such as switch 25). The actuator causes delivery of the denervating agent through the lumens of each of needles 34, either by opening a valve, activating a pump, or a combination of both. In either case, the plurality of needles 34 allow for simultaneous delivery of doses of the denervating agent at specific different prostate locations. Such simultaneous delivery of the denervating agent can simplify the procedure and reduce patient trauma by avoiding unnecessary movement and rotation of shaft 22B within the urethra, as well as multiple steps for puncture of the prostate. In addition, delivery of the denervating agent at precise locations may reduce the amount of the denervating agent needed for effective therapeutic results.

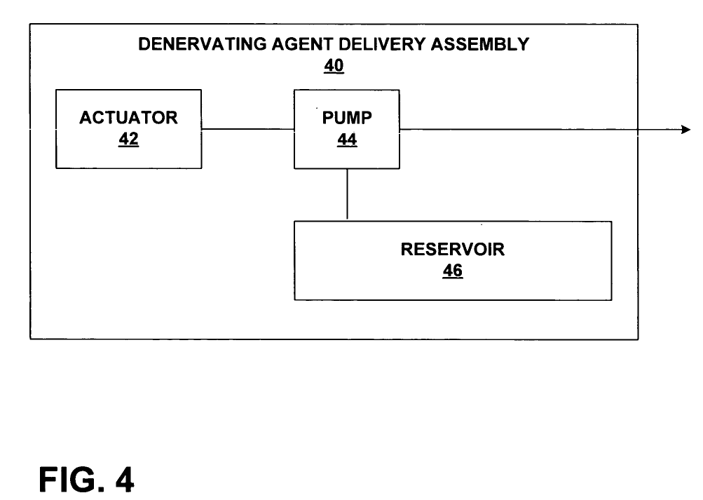

FIG. 4 is a block diagram of one exemplary denervating agent delivery assembly 40 which may be used with one or more devices or systems described herein. As shown, denervating agent delivery assembly 40 includes an actuator 42, a pump 44, and a reservoir 46. When a physician actuates actuator 42, control signals are sent to pump 44. Pump 44 causes a denervating agent to flow from reservoir 46, through the lumen of one or more needles, and into the prostate gland of a patient. In some embodiments, the actuation of actuator 42 causes a discrete dose to be delivered, and in other cases the denervating agent is delivered in a continuous fashion as long as actuator 42 is actuated. In some cases, settings can be established such that actuation of actuator 42 causes delivery of a defined dosage, reducing the possibility for human error in delivering the dosage. The dosages may be defined by the physician and possibly changed for delivery to different locations. In this manner, the physician can delivery a precise dosage of the denervating agent or selectively control the amount of the denervating agent delivered with each dosage. Denervating agent delivery assembly 40 may correspond to assembly 19 (FIG. 1) and in that case, actuator 42 would correspond to switch 25. Alternatively, denervating agent delivery assembly 40 may be used with other systems described in greater detail below.

In some cases, when multiple delivery needles 34 are used, such as illustrated in FIG. 3, a separate actuator and pump may be used to cause discrete delivery of the denervating agent through each needle. However, the same actuator, pump and reservoir could also be used for multiple needles. In the latter case, however, pressure regulation through the different needles would be more difficult. Thus, the use of separate reservoirs and pumps may be advantageous when multiple needles are used, in order to simplify the control of dosage delivery of the denervating agent. Also, separate reservoirs may allow for delivery of different denervating agents via different needles. Alternatively, a single pump with separate reservoirs may be used for the needles.

Multiple reservoirs could also be used with each individual needle. For example, a first reservoir may hold a substantial amount of the denervating agent, whereas a second reservoir may hold a discrete dose of the denervating agent. In that case, actuation of actuator 42 could cause pump 44 to deliver the discrete dose from the second reservoir. Following actuation of actuator 42, the second reservoir could be reloaded with another dose from the first reservoir, e.g. via another pump. Other variations or modifications of denervating agent delivery assembly 40 could also be used.

The denervating agent may comprise a botulinum toxin such as botulinum toxin type A (commercially available from Allergan of Irvine, Calif., and sold under the trade name BOTOX®), although the invention is not necessarily limited in that respect. Other denervating agents that may be used include capsaicin, resinoferatoxin, alpha-bungotoxin, or other agents that are generally toxic to mammalian nervous systems. In some cases, the denervating agent may be generally non-toxic to mammalian muscle systems or other non-neural anatomy. In other cases, however, the denervating agent may cause debulking or necrossing effects to muscle tissue.

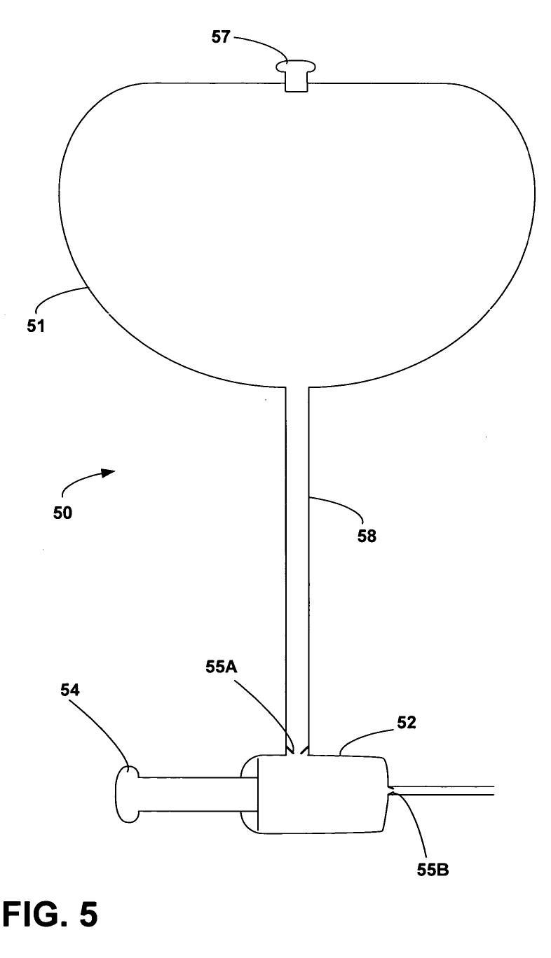

FIG. 5 is a conceptual side view of one exemplary denervating agent delivery assembly 50 which may be used with one or more devices or systems described herein. As shown, denervating agent delivery assembly 50 includes a first reservoir 51 that holds a substantial amount of the denervating agent. First reservoir 51 may include a cap 57 that can be removed to refill first reservoir with the denervating agent. A second reservoir 52 holds a discrete dose of the denervating agent. By way of example, first reservoir 51 may hold greater than approximately 4 milliliters of the botulinum toxin, and second reservoir may hold less than approximately 1 milliliter of the botulinum toxin, such as a dose of approximately 0.3-0.7 milliliter of botulinum toxin, more preferably approximately 0.4-0.6 milliliter of botulinum toxin, and still more preferably approximately 0.5 milliliter of botulinum toxin. Again, these dosages may include a diluent of approximately 0.9 percent sodium chloride in saline, resulting in dosages that include between approximately 1.25 to 10 units of botulinum toxin per 0.1 milliliter.

First reservoir 51 and second reservoir 52 may be mechanically coupled via a hose 58 or other type of fluid line. An actuator 54 is mechanically coupled to second reservoir 52 and servers to deliver the discrete dose within second reservoir 52 through a lumen of one or more needles. For example, actuator 54 may comprise a manual or automated plunger mechanism that mechanically forces the denervating agent from second reservoir 52 through a lumen of one or more needles.

Following actuation of actuator 54 second reservoir 52 refills with another dose of the denervating agent for subsequent delivery. A system of valves 55A, 55B may ensure that when actuator 54 is depressed, the denervating agent flows from second reservoir 52 through a lumen of one or more needles, and when actuator recoils, second reservoir 52 refills with another dose of the denervating agent from first reservoir 51. For example, valve 55A may comprise a check valve with a valve poppet that unseats under negative pressure from withdrawal of actuator 54, and valve 55B may comprise a check valve with a valve poppet that unseats under positive pressure from activation of actuator 54. Other valve arrangements could also be used.

In some cases, when multiple delivery needles are used, such as illustrated in FIG. 3, a set of second reservoirs (similar to reservoir 52) may be used respectively for each needle. In that case, the set of second reservoirs would be mechanically coupled to a first reservoir that holds a substantial amount of the denervating agent. Alternatively, a set of needles can be coupled to the same second reservoir and one dose would be dispersed through the various needles.

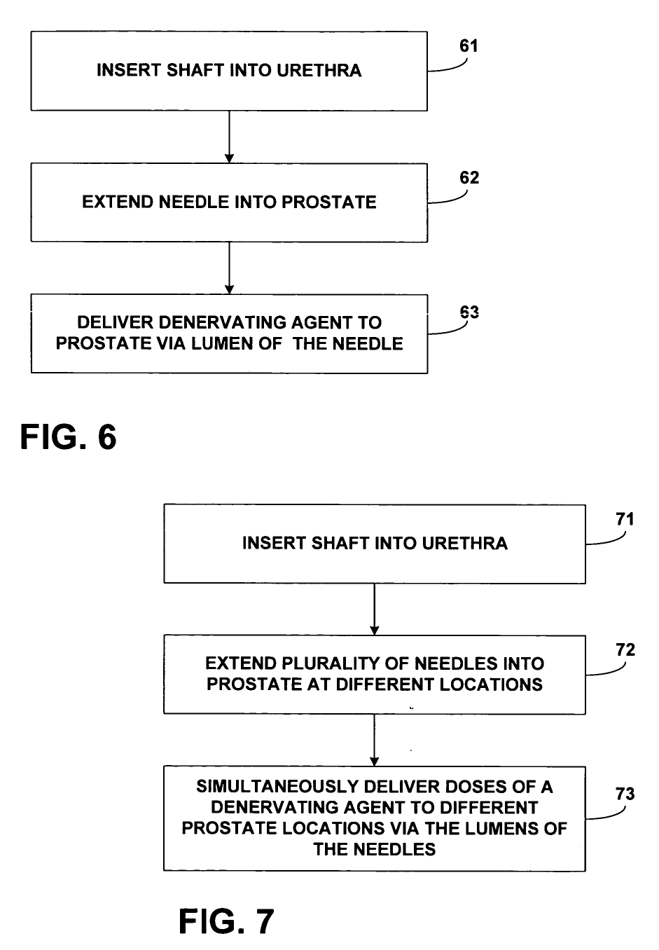

FIG. 6 is a flow diagram illustrating a transurethral technique for delivery of a denervating agent to the prostate gland according to an embodiment of the invention. As shown in FIG. 6, a physician inserts shaft 20 into a urethra of a patient in proximity to a prostate gland of the patient (61). For example, an endoscope 28 may be housed within a substantially transparent distal tip 27 of shaft 20A, and the physician may guide shaft 20A in proximity to the prostate gland using images generated by endoscope 28 and displayed on endoscopic output equipment 18. In order to aid the physician's ability to navigate shaft 20A through the urethra of the patient, distal tip 22A of shaft 20A may define an offset curvature as described above.

Once distal tip 22A of shaft 20A is in proximity to the prostate gland, needle 38 is extended into the prostate gland (62). For example, the physician may actuate slider bar 23 (FIG. 1) to cause needle 38 (FIG. 2) to advance forward and extend from the side of shaft 20A. Needle 38 may be spring-loaded in that slide bar 23 tends to spring forward to spring-bias needle 38 into the prostate gland, helping to ensure that needle 38 will pierce the prostate tissue. One or more doses of a denervating agent can then be delivered to the prostate gland via lumen 29 of needle 38 (63). Again, the denervating agent may comprise, for example, botulinum toxin. In this manner, treatment of BPH or other prostate disorders can be realized.

FIG. 7 is another flow diagram illustrating another transurethral technique for delivery of a denervating agent to the prostate gland according to an embodiment of the invention. As shown in FIG. 7, a physician inserts shaft 20 into a urethra of a patient to proximity of a prostate gland of the patient (71). Again, an endoscope housed within the shaft may be used by the physician to aid guidance of the shaft in proximity to the prostate gland, and the distal tip of the shaft may also be shaped with an offset curvature to improve navigation through the urethra of the patient.

Once distal tip 22B of shaft 20B is in proximity to the prostate gland, a plurality of needles 34 are extended into the prostate gland from the side of shaft 20B to pierce the prostrate gland in a plurality of locations (72). The different locations may, for example, correspond to different lobes of the prostate gland, although the invention is not necessarily limited in that respect. Doses of the denervating agent can be delivered simultaneously to the different prostate locations via respective lumens of needles 34 (73). In this manner, delivery of the denervating agent can be performed quickly in a targeted manner, possibly reducing the likelihood of complication.

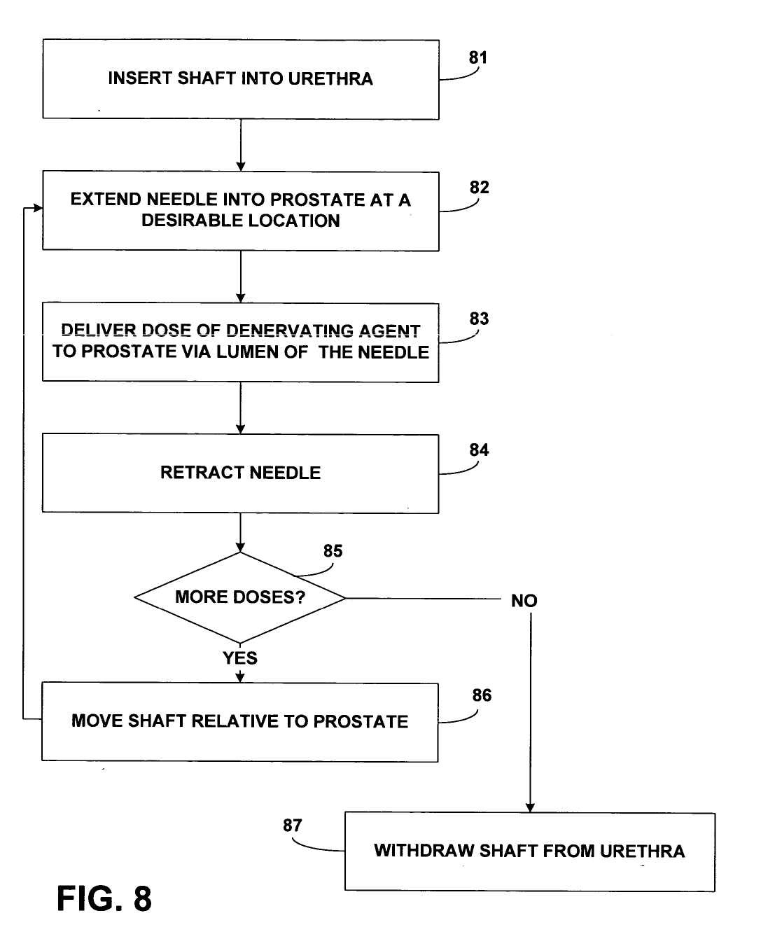

FIG. 8 is another flow diagram illustrating another transurethral technique for delivery of a denervating agent to the prostate gland according to an embodiment of the invention. As shown in FIG. 8, a physician inserts shaft 20 into a urethra of a patient to proximity of a prostate gland of the patient (81). Again, an endoscope 28 may be housed within a substantially transparent distal tip 27 of shaft 20A, and the physician may guide shaft 20A in proximity to the prostate gland using images generated by endoscope 28 and displayed on endoscopic output equipment 18. Also, in order to aid the physician's ability to navigate shaft 20A through the urethra of the patient, shaft 20A the distal tip 22A of shaft 20A may define an offset curvature.

Once distal tip 22A of shaft 20A is in proximity to the prostate gland, needle 38 is extended into the prostate gland at the desired location (82). For example, the physician may actuate slide bar 23 to cause needle 38 to extend from the side of shaft 20A. Needle 38 may be spring-loaded in that slide bar 23 tends to spring forward to spring-bias needle 38 into the prostate gland, helping to ensure that needle 38 will pierce the prostate tissue. A dose of a denervating agent can then be delivered to the prostate gland via lumen 29 of needle 38 (83). Again, the denervating agent may comprise botulinum toxin or another denervating agent.

The physician then retracts needle 38 (84), for example, by moving slide bar 23. If needle 38 is spring-loaded, the physician may need to exert pressure on slide bar 23 to retract and lock needle 38 in a retracted position. If more doses are desired (yes branch of 85), the physician moves shaft 20A relative to the prostate gland (86), and then extends the needle to pierce the prostrate gland in a second location (83). The physician may use endoscopic output to facilitate such repositioning of the shaft relative to the prostate gland.

The physician may continue by extending needle 38, delivering a dose of the denervating agent via lumen 29, and then retracting needle 38 and repositioning shaft 20A until additional doses are unnecessary (no branch of 85). At that point, the physician can withdraw shaft 20A from the patient's urethra (87). Advantageously, device 10 described above, allows the physician to deliver a plurality of doses of the denervating agent to different prostate locations, e.g., different lobes, without removing shaft 20A until all the doses have been delivered. Any number of doses may be delivered in accordance with the invention, prior to withdrawing shaft 20A from the urethra. In this manner, the targeted and localized delivery of the denervating agent to specific prostate locations may improve treatment of BPH or other prostate disorders. Needle 38 may be advanced to different depths, for each dose, based on the given location where the denervating agent is being delivered. Moreover, the size of the dosages may vary for the different locations.

By way of example, each of the doses may comprise approximately 0.3-0.7 milliliter of botulinum toxin, more preferably approximately 0.4-0.6 milliliter of botulinum toxin, and still more preferably approximately 0.5 milliliter of botulinum toxin. The total number of doses may be less than 10 over the course of a single procedure. For example, the total number of doses may be greater than one and less than eight with dosages less than approximately 0.5 milliliter of botulinum toxin. Accordingly, less than 4 milliliters of botulinum toxin may be delivered in a targeted fashion to different prostate locations, which may improve the therapeutic effect.

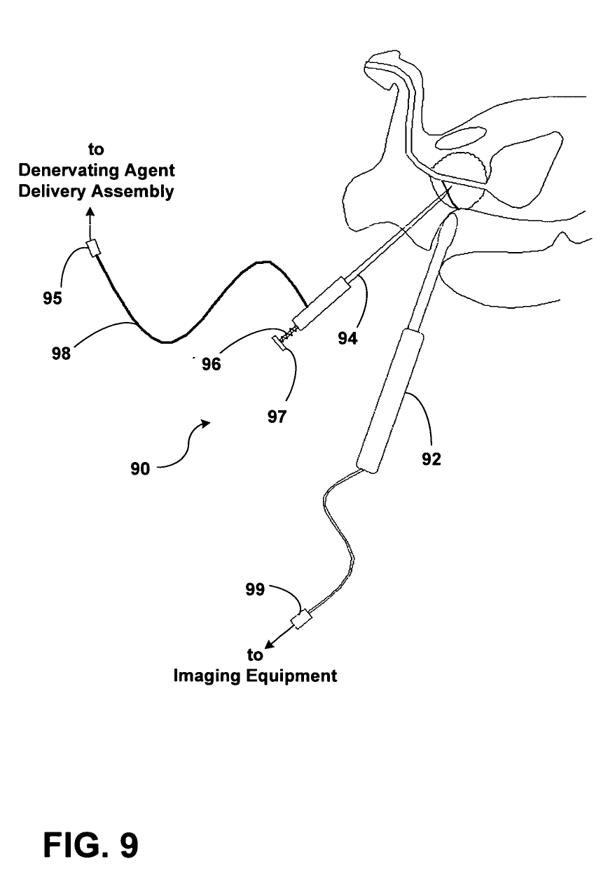

FIG. 9 is a conceptual cross-sectional side view of a system 90 that may be used in a transperineal technique for delivery of a denervating agent to the prostate gland according to an embodiment of the invention. As shown in FIG. 9, system 90 includes an imaging apparatus 92 sized for insertion into a rectum of a patient to generate one or more images of a prostate gland. For example, imaging apparatus 92 may comprise an ultrasonic imaging probe similar to one of the LOGIQ 500/400 PRO Series or LOGIQ 700 EXPERT/PRO Series, commercially available from GE Medical Systems of Waukesha, Wis.

Imaging apparatus 92 may comprise an ultrasonic transrectal end-firing probe, a true transverse/axial probe, a true longitudinal/sagittal probe, a biplane probe, or any other suitable imaging apparatus that uses ultrasonic or other imaging techniques. If imaging apparatus 92 is an ultrasonic probe, it may operate in the 5-9 MHz range or another range. In that case, needle 94 may include a hyper-echoic coating for improved ultrasonic viewability.

Imaging apparatus 92 may be coupled to imaging equipment, which displays the output generated by imaging apparatus 92. For example, a communication interface 99 may facilitate communicative coupling between imaging apparatus 92 and the imaging equipment. Suitable imaging equipment includes standard ultrasonic imaging equipment, also commercially available from GE Medical Systems of Waukesha, Wis.

System 90 also includes a needle 94 for insertion through a perineum of the patient in proximity to the prostate gland based on one or more images generated by imaging equipment. Needle 94 defines a lumen through which a denervating agent can be delivered to the prostate gland. A hub 95 can facilitate attachment of needle 94 to allow attachment of needle 94 to a denervating agent delivery assembly, such as an assembly similar to that illustrated in either of FIG. 4 or 5. An optional fluid line 98 may provide fluid communication between hub 95 and needle 94.

System 90 also includes a spring mechanism 96 to bias needle 94 into the prostate gland upon actuation. In other words, a physician can insert needle into proximity to the prostate gland and then actuate spring mechanism 96 to cause needle 94 to bias into the prostate gland to that a denervating agent can be delivered to the prostate gland through the lumen of needle 94. Spring mechanism 96 helps ensure that needle 94 will properly pierce the prostate gland. Actuator 97 facilitates actuation of spring mechanism 96 by the physician and may comprise a button, or the like. The physician presses actuator 97 which causes spring mechanism 96 to bias needle 94 into the prostate gland of the patient. Needle 94 may also be advancable to different depths, if desired, e.g. by incorporating an adjustment instrument with spring mechanism 96.

After delivering a dose of the denervating agent to a first location of the prostate gland, the physician may retract needle 94 by either pulling on needle 94 or retracting actuator 97 to reset spring mechanism 96. The physician may then reposition needle 94 with respect to the prostate gland and actuate spring mechanism 96 to cause needle 94 to pierce the prostate gland in another location for delivery of a second dose. This process can be repeated for a plurality of doses, with each dosage conforming to the size and amounts described herein. Imaging apparatus 92 can ensure that needle 94 is precisely positioned for the delivery of the doses of the denervating agent to the appropriate prostate locations.

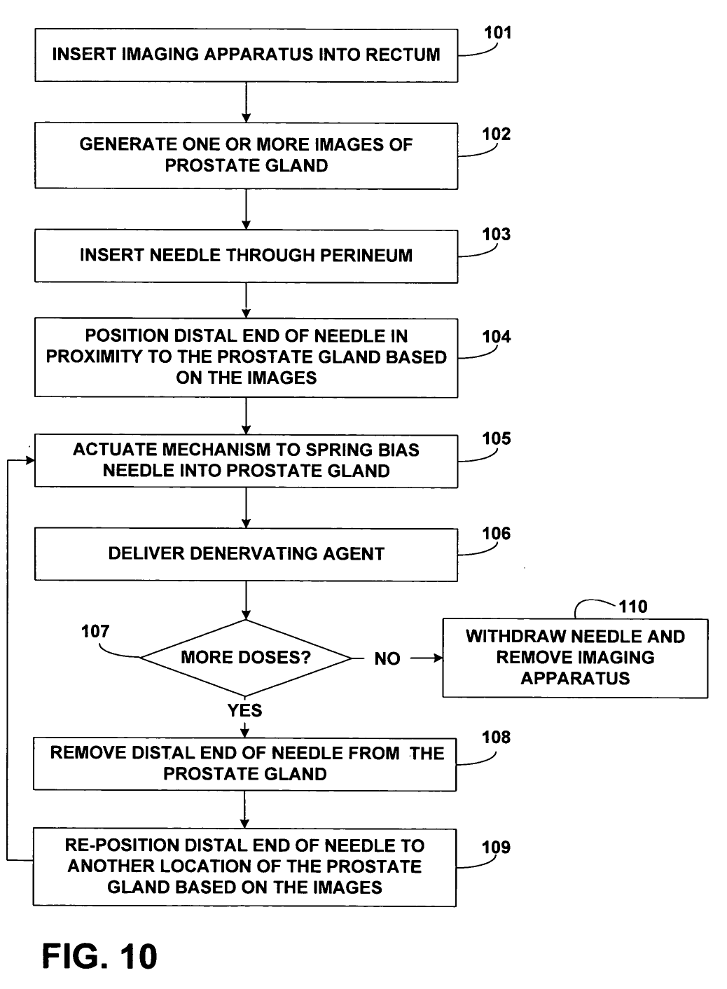

FIG. 10 is a flow diagram illustrating a transperineal technique for delivering a denervating agent to the prostate gland. As shown, the physician inserts imaging apparatus 92 into the rectum of the patient (101), and using imaging apparatus 92, generates one or more images of the prostate gland of the patient (102). For example, the physician may maneuver imaging apparatus 92 to generate images that are displayed on imaging equipment communicatively coupled to imaging apparatus 92.

The physician then inserts needle 94 through the perineum of the patient (103), and positions a distal end of needle 94 in proximity to the prostate gland based on the images generated by imaging apparatus 92 (104). In order to pierce the prostate gland, the physician actuates spring mechanism 96 by pressing actuator 97, causing needle 94 to spring bias into the prostate gland (105). A denervating agent is delivered to the prostate gland via a lumen of needle 94 (106). For example, hub 95 may be attached to a deneravating agent delivery assembly that the physician can actuate to cause the denervating agent to flow through the lumen of needle 94 and into the prostate gland.

If desired, system 90 can be used to deliver a plurality of doses of the denervating agent. If more doses are desired (yes branch of 107), the physician can remove the distal end of needle 94 from the prostate gland (108) and re-position the distal end of needle 94 to another location of the prostate gland based on the images generated by imaging apparatus 92 (109). In particular, the physician may completely remove needle 94 from the perineum and then reinsert needle 94 to another location, or may simply withdraw needle 94 from the prostate gland, e.g., by re-cocking actuator 97 to re-load spring mechanism 96. In any case, once the distal end of needle 94 is re-positioned to another location of the prostate gland, the physician can again actuate spring mechanism 96 by pressing actuator 97, thereby causing needle 94 to spring bias into the prostate gland (105). Another dose of the denervating agent is then delivered to the prostate gland at the new location via a lumen of needle 94 (106).

This process of repeating doses can be repeated a number of times to deliver doses to a first location, a second location, a third location, a fourth location, and so forth. Each dose, for example, may comprise approximately 0.3-0.7 milliliter of botulinum toxin, more preferably approximately 0.4-0.6 milliliter of botulinum toxin, and still more preferably approximately 0.5 milliliter of botulinum toxin. The denervating agent delivery assemblies described above with reference to FIGS. 4 and 5 may facilitate precise delivery of discrete doses, e.g., according to an indexed pumped advancement of the denervating agent, or discrete dosages defined by the size of a mechanical delivery reservoir. Again, the size of the dosages may be programmed into the pump such that actuation cause delivery of a defined dosage, and may also be changed by the physician, e.g., for delivery at different prostate locations. Once the desired doses are delivered, needle 94 can be withdrawn from the patient's perineum and imaging device 92 can be removed from the patient's rectum (110).

Again, the targeted and localized delivery of the denervating agent to specific prostate locations may improve treatment of BPH or other prostate disorders. By way of example, less than ten doses of less than approximately 0.5 milliliter of botulinum toxin can be delivered. More specifically, the total number of doses may be greater than one and less than eight. Accordingly, less than 4 milliliters of botulinum toxin may be delivered in targeted fashion to different prostate locations, which may improve the therapeutic effect.

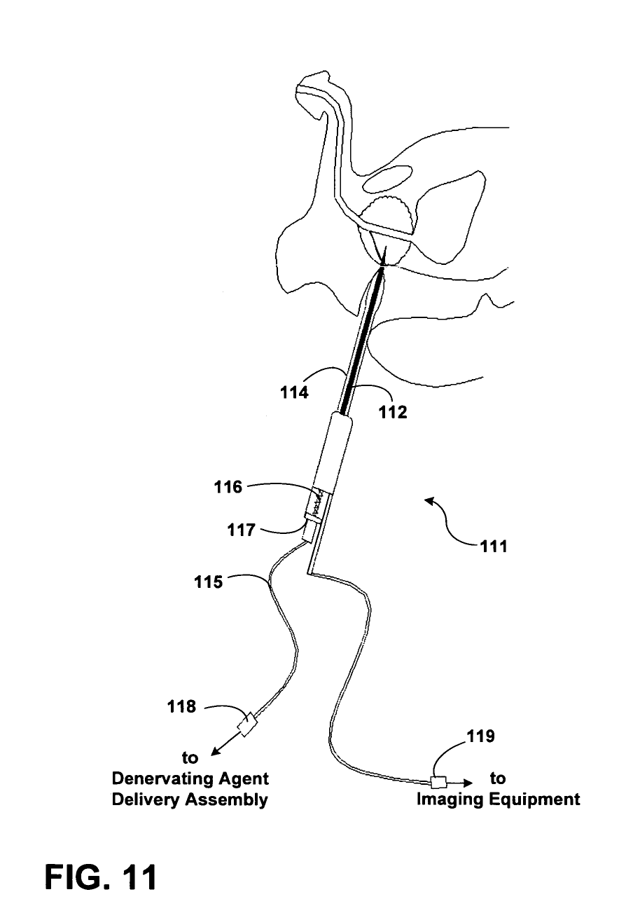

FIG. 11 is a conceptual cross-sectional side view of a system 111 that may be used in a transrectal technique for delivery of a denervating agent to the prostate gland according to an embodiment of the invention. As shown in FIG. 11, system 111 includes an imaging apparatus 114 sized for insertion into a rectum of a patient to generate one or more images of a prostate gland. As further shown in FIG. 11, imaging apparatus 114 is formed with a hole. Needle 112 is positioned through the hole of imaging apparatus 114. The hole through imaging apparatus 114, for example, extends along a longitudinal length of apparatus 114 and through a distal tip of imaging apparatus 114. Needle 112 mates with the hole formed through imaging apparatus 114 and is moveable in the longitudinal direction such that needle can be extended from the distal tip of imaging apparatus 114 through the hole.

For example, imaging apparatus 114 can comprise a probe-shaped body defining a major longitudinal direction. A hole may be formed through the probe-shaped body along the major longitudinal direction, e.g., corresponding to the location of needle 112 through imaging apparatus 114, as illustrated in FIG. 11. In other words, the hole through imaging apparatus 114 is sized to mate with a fluid delivery needle, such as needle 114, so that when imaging apparatus 114 images a location of a patient, e.g., the prostate gland from inside the patient's rectum, needle 112 can be extended through the hole and out a distal end of imaging apparatus 114 to pierce the patient at the location, e.g., at the prostate gland.

Imaging apparatus 114 can be pressed against the rectal wall of the patient in order to image the location of the patient's prostate gland. Needle 112 can be advanced through the hole and out the distal end of imaging apparatus 114. Accordingly, needle 112 can be advanced to pierce through the rectal wall of the patient in proximity to the prostate gland based on the one or more images generated by imaging device. Needle 112 defines a lumen such that a denervating agent can be delivered to the prostate gland through the lumen.

For example, imaging apparatus 114 may comprise an ultrasonic imaging probe similar to one of the LOGIQ 500/400 PRO Series or LOGIQ 700 EXPERT/PRO Series, commercially available from GE Medical Systems of Waukesha, Wis. However, imaging apparatus 114 would be substantially different than such commercially available probes in that imaging apparatus 114 defines the hole through which needle 112 mates, as shown in FIG. 11. Imaging apparatus 114 may comprise an ultrasonic transrectal end-firing probe, a true transverse/axial probe, a true longitudinal/sagittal probe, a biplane probe, or any other suitable imaging apparatus that uses ultrasonic or other imaging techniques. If imaging apparatus 114 is an ultrasonic probe, it may operate in the 5-9 MHz range or another range. Again, however, in order to facilitate transrectal denervating agent delivery, imaging apparatus 114 includes a hole through which needle 112 can be advanced through imaging apparatus 114, out a distal tip of imaging apparatus 114, into the patient's rectal wall and into the patient's prostate gland as shown in FIG. 11.

The hole formed through imaging apparatus 114 may have a diameter approximately similar to the diameter of needle 114, or may define a diameter be slightly larger than that of needle 114. If desired, imaging apparatus 114 and needle 112 may include surface variations formed to facilitate improved mechanical interaction between imaging apparatus 114 and needle 114, when needle is maneuvered through the hole. For example, a protrusion on needle 112 may interact with a channel formed in the hole of imaging apparatus 114 in order to improve mechanical guidance of needle 112 through the hole in imaging apparatus 114. Also, in some cases, surface variations formed on needle 112 and in the hole of imaging apparatus 114 may help maintain assembly of needle 112 within the hole of imaging apparatus 114, e.g., when system 111 is not in use. The surface variations are subject to a wide variety of possible implementations and can generally improve interaction between the components of system 111 for guiding or to maintain interlocking of the components of system 111. In addition, needle 112 may include a hyper-echoic coating to improve viewability of needle 112 by imaging apparatus 114.

Imaging apparatus 114 may be coupled to imaging equipment, which displays the output generated by imaging apparatus 114. For example, a communication interface 119 may facilitate communicative coupling between imaging apparatus 114 and the imaging equipment. Suitable imaging equipment includes standard ultrasonic imaging equipment, also commercially available from GE Medical Systems of Waukesha, Wis. Other imaging equipment, of course, would be used if imaging apparatus 114 were to use other imaging technology.

In some transrectal embodiments, system 111 may further include a spring mechanism 116 to bias needle into the prostate gland upon actuation. A physician can insert needle 112 through the hole formed in imaging apparatus 114, out a distal end of imaging apparatus, through the rectal wall of the patient, and into proximity to the prostate gland. The physician may then actuate spring mechanism 116 to cause needle 112 to bias into the prostate gland to that a denervating agent can be delivered to the prostate gland through the lumen of needle 114. Spring mechanism 116 helps ensure that needle 112 will properly pierce the prostate gland. Actuator 117 facilitates actuation of spring mechanism 116 by the physician and may comprise a button, or the like. The physician presses actuator 117 which causes spring mechanism 116 to bias needle 112 into the prostate gland of the patient. When retracted, actuator 117 may lock spring mechanism 116 in a spring-loaded configuration such that when pressed, actuator 117 causes spring mechanism 116 to exert its spring potential on needle 112 to bias needle 112 into the prostate gland. Needle 112 may also be advancable to different depths, if desired, e.g. by incorporating an adjustment instrument with spring mechanism 116.

As mentioned, needle 112 defines a lumen through which the denervating agent can be delivered to the prostate gland. A hub 118 can facilitate attachment of needle 112 to a denervating agent delivery assembly, such as an assembly similar to that illustrated in either of FIG. 4 or 5. An optional fluid line 115 may provide fluid communication between hub 118 and needle 114.

After delivering a dose of the denervating agent to a first location of the prostate gland, the physician may retract needle 112 by either pulling on needle 112 or retracting actuator 117 to reset spring mechanism 116. The physician may then reposition needle 112 with respect to the prostate gland and actuate spring mechanism 116 to cause needle 112 to pierce the prostate gland in another location for delivery of a second dose. This process can be repeated for a plurality of doses. Imaging apparatus 114 can ensure that needle 112 is precisely positioned for the delivery of the doses of the denervating agent to the appropriate prostate locations in accordance with a transrectal technique.

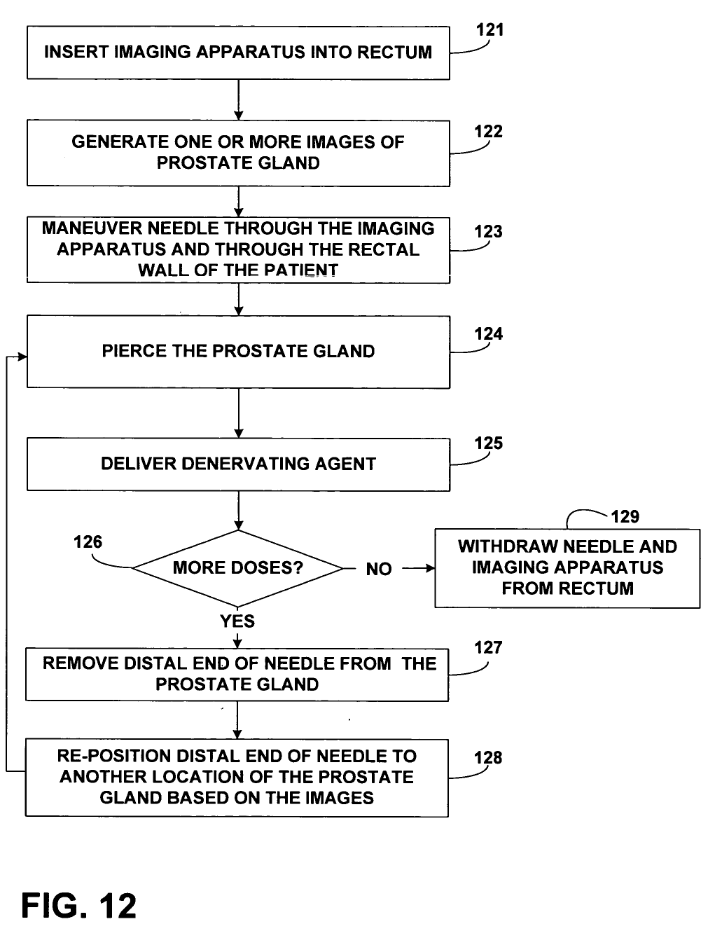

FIG. 12 is a flow diagram illustrating a transrectal technique for delivering a denervating agent to the prostate gland. As shown, the physician inserts imaging apparatus 114 into the rectum of the patient (121), and using imaging apparatus 114, generates one or more images of the prostate gland of the patient (122). For example, the physician may maneuver imaging apparatus 114 to generate images that are displayed on imaging equipment communicatively coupled to imaging apparatus 114.

The physician then maneuvers needle 112 through imaging apparatus 114 and through a rectal wall of the patient (123). For example, needle 112 may be pre-assembled through the hole formed in imaging apparatus 114, e.g., prior to insertion of imaging apparatus 114 into the patient's rectum, or may be inserted through the hole after imaging apparatus 114 is inserted into the patient's rectum. In either case, the distal end of needle 112 is caused to pierce the prostate gland (124), and a denervating agent is delivered to the prostate gland via a lumen of needle 112 (125). For example, hub 119 may be attached to a denervating agent delivery assembly (such as that illustrated in FIG. 4 or FIG. 5) to provide fluid communication between the denervating agent delivery assembly and needle 114. The physician may actuate an actuator of the denervating agent delivery assembly to cause the denervating agent to flow through the lumen of needle 112 and into the prostate gland.

As mentioned, in some transrectal embodiments, system 111 may further include a spring mechanism 116 to bias needle into the prostate gland upon actuation. In that case, inserting the distal end of needle 112 into the prostate gland may comprise actuating spring mechanism 116 to cause the distal end of the needle to spring bias into the prostate gland.

If desired, system 111 can be used to deliver a plurality of doses of the denervating agent. If more doses are desired (yes branch of 126), the physician can remove the distal end of needle 112 from the prostate gland (127) and re-position the distal end of needle 112 to another location of the prostate gland based on the images generated by imaging apparatus 114 (128). In particular, the physician may completely remove needle 112 the rectal wall and then reinsert needle 112 to another location, or may simply withdraw needle 112 from the prostate gland and reposition needle 112 to another location without fully withdrawing needle 112 from the rectal wall. In any case, once the distal end of needle 112 is re-positioned to another location of the prostate gland, the physician can pierce the prostate gland with needle 112 in another location (124). Another dose of the denervating agent is then delivered to the prostate gland at the new location via a lumen of needle 112 (125).

This process of repeating doses can be repeated a number of times to deliver doses to a first location, a second location, a third location, a fourth location, and so forth. Each dose, for example, may comprise approximately 0.5 milliliter of botulinum toxin. The denervating agent delivery assemblies described above with reference to FIGS. 4 and 5 may facilitate precise delivery of discrete doses, e.g., according to an indexed advancement of the denervating agent or discrete dosages defined by the size of a mechanical delivery reservoir. Once the desired doses are delivered, needle 112 and imaging apparatus 114 can be withdrawn from the patient's rectum (129).

Again, the targeted and localized delivery of the denervating agent to specific prostate locations may improve treatment of BPH or other prostate disorders. By way of example, less than ten doses of approximately 0.5 milliliter of botulinum toxin can be delivered. More specifically, the total number of doses may be greater than one and less than eight. Accordingly, an overall dosage of less than 4 milliliters of botulinum toxin may be delivered in targeted fashion to different prostate locations, in a series of smaller dosages, which may improve the therapeutic effect.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using devices and systems for delivery of a denervating agent, as described herein. As used herein, the term patient refers to any animal that includes a prostate gland, i.e. male animals. Put another way, the same techniques and devices described herein may also be useful for human or non-human patients.

In many of the described embodiments, the denervating agent is described as a botulinum toxin such as botulinum toxin type A (commercially available from Allergan of Irvine, Calif. and sold under the trade name BOTOX®). Other denervating agents, however, may also be used such as capsaicin, resinoferatoxin, alpha-bungotoxin, or other agents that are generally toxic to mammalian nervous systems. In some cases, the denervating agent may be generally non-toxic to mammalian muscle systems or other non-neural anatomy. In other cases, however, the denervating agent may also necross or debulk mammalian muscle tissue. If BOTOX® is used, dosages may include a diluent of approximately 0.9 percent sodium chloride in saline, resulting in dosages that include between approximately 1.25 to 10 units of botulinum toxin per 0.1 milliliter.

Also, although many of the techniques described herein have been described as being therapeutic for treatment of BPH, they may prove useful for any of a wide variety of other prostate disorders. In addition, combinations of the transurethral, transperineal and transrectal techniques may be desirable in order to facilitate delivery of denervating agents to a wider variety of prostate locations. In other words, a medical procedure may include combinations or sub-combinations of the various techniques described herein.

Moreover, the an imaging apparatus comprising a probe-shaped body defining a major longitudinal direction, and hole formed through the probe-shaped body along the major longitudinal direction, as described with reference to FIG. 1, may be useful for other non-prostate imaging, e.g., whenever it is desirable to advance a needle at the location of imaging.

In the appended claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for delivering a denervating agent to a prostate gland, the method comprising:
inserting an imaging apparatus into a rectum of a patient;
generating one or more images of the prostate gland via the imaging apparatus;
inserting a needle through a perineum the patient;
positioning a distal end of the needle in proximity to the prostate gland based on the one or more images;
actuating a mechanism to cause the distal end of the needle to spring bias into the prostate gland; and
delivering the denervating agent to the prostate gland via a lumen of the needle.

2. The method of claim 1, wherein the imaging apparatus comprises an ultrasonic imaging device.

3. The method of claim 2, further comprising maneuvering the ultrasonic imaging device to generate the one or more images of the prostate gland.

4. The method of claim 1, wherein the denervating agent includes botulinum toxin.

5. The method of claim 1, further comprising:
actuating the mechanism to cause the distal end of the needle to spring bias into the prostate gland at a first location;
delivering a first dose of the denervating agent to the prostate gland via the lumen of the needle;
removing the distal end of the needle from the prostate gland at the first location;
positioning the distal end of the needle in proximity to a second location of the prostate gland based on the one or more images;
actuating the mechanism again to cause the distal end of needle to spring bias into the prostate gland at the second location; and
delivering a second dose of the denervating agent to the prostate gland via the lumen of the needle.

6. The method of claim 5, further comprising:
removing the distal end of the needle from the prostate gland at the second location;
positioning the distal end of the needle in proximity to a third location of the prostate gland based on the one or more images;
actuating the mechanism again to cause the distal end of needle to spring bias into the prostate gland at the third location; and
delivering a third dose of the denervating agent to the prostate gland via the lumen of the needle.

7. The method of claim 6, further comprising:
removing the distal end of the needle from the prostate gland at the third location;
positioning the distal end of the needle in proximity to a fourth location of the prostate gland based on the one or more images;
actuating the mechanism again to cause the distal end of needle to spring bias into the prostate gland at the fourth location; and
delivering a fourth dose of the denervating agent to the prostate gland via the lumen of the needle.

8. The method of claim 7, wherein each of the doses comprise approximately 0.5 milliliter of botulinum toxin.

9. The method of claim 1, further comprising delivering the denervating agent from a denervating agent delivery assembly that includes a reservoir to hold the denervating agent and an actuator to cause the denervating agent to flow from the reservoir through the lumen, wherein a hub and a fluid line attaches the reservoir to the needle.

10. The method of claim 1, further comprising delivering the denervating agent from a denervating agent delivery assembly that includes a first reservoir that holds a substantial amount of the denervating agent, a second reservoir to hold a first discrete dose of the denervating agent and an actuator to cause the denervating agent to flow from the second reservoir through the lumen, wherein a hub attaches the second reservoir to the needle and the second reservoir refills with a second discrete dose of the denervating agent from the first reservoir following delivery of the first discrete dose.

11. A system for delivering a denervating agent to a prostate gland comprising:
an imaging apparatus sized for insertion into a rectum of a patient to generate one or more images of a prostate gland;
a needle for insertion through a perineum of the patient in proximity to the prostate gland based on the one or more images, the needle defining a lumen; and
a spring mechanism to bias the needle into the prostate gland upon actuation such that a denervating agent can be delivered to the prostate gland through the lumen.

12. The system of claim 11, further comprising an actuator to actuate the spring mechanism to bias the needle into the prostate gland.

13. The system of claim 11, further comprising a denervating agent delivery assembly coupled to the needle to deliver the denervating agent through the lumen.

14. The system of claim 13, wherein the denervating agent delivery assembly includes a reservoir to hold the denervating agent and an actuator to cause the denervating agent to flow from the reservoir through the lumen.

15. The system of claim 14, wherein the second actuator comprises a plunger.

16. The system of claim 14, wherein further comprising a hub and a fluid line for attachment of the needle to the reservoir.

17. The system of claim 13, wherein the denervating agent delivery assembly includes a first reservoir to hold a substantial amount of the denervating agent, a second reservoir to hold a discrete dose of the denervating agent, and an actuator to cause the denervating agent to flow from the second reservoir through the lumen, wherein the second reservoir refills with another discrete dose of the denervating agent from the first reservoir following actuation of the second actuator.

18. The system of claim 13, wherein the denervating agent delivery assembly includes a second actuator, a pump and a reservoir, wherein upon actuation of the second actuator the pump causes delivery of the denervating agent from the reservoir through the lumen.

19. The system of claim 11, wherein the denervating agent includes botulinum toxin.

20. The system of claim 11, wherein the imaging apparatus comprises an ultrasonic imaging apparatus.

21. The system of claim 11, wherein the needle includes a hyper-echoic coating.

22. A system for delivering a denervating agent to a prostate gland comprising:
an imaging apparatus sized for insertion into a rectum of a patient to generate one or more images of a prostate gland;
a needle for insertion through a perineum of the patient in proximity to the prostate gland based on the one or more images, the needle defining a lumen; and
means for spring-biasing the needle into the prostate gland such that a denervating agent can be delivered to the prostate gland through the lumen.

23. The system of claim 22, further comprising means for delivering the denervating agent through the lumen.
